(19) United States
(12) Patent Application Publication
(10) Pub. No.: US 2024/0302420 A1
(43) Pub. Date: Sep. 12, 2024

(54) SYSTEMS AND METHODS FOR EVALUATION OF TRANSCRANIAL MAGNETIC STIMULATION INDUCED ELECTRIC FIELDS

(71) Applicant: The General Hospital Corporation, Boston, MA (US)

(72) Inventors: Aapo Nummenmaa, Boxford, MA (US); Mohammad Daneshzand, Boston, MA (US); Sergey N. Makarov, Holden, MA (US)

(21) Appl. No.: 18/546,826

(22) PCT Filed: Feb. 17, 2022
(86) PCT No.: PCT/US2022/016833

§ 371 Date: Nov. 29, 2023

Related U.S. Application Data
(60) Provisional application No. 63/150,549, filed on Feb. 17, 2021.

(51) Int. Cl. G01R 29/12; A61N 2/00; A61N 2/02

(52) U.S. Cl. CPC G01R 29/12; A61N 2/006; A61N 2/02

(57) ABSTRACT

Systems, methods, and media for E-field determination are provided. In some embodiments, a method for E-field determination for an electromagnetic coil positioned about a subject having one or more conductivity boundaries comprises: retrieving a predetermined electromagnetic coil E-field map, boundary model, and Magnetic Stimulation Profile (MSP), wherein the MSP comprises the incident E-field at a surface of interest (A_{inc}) caused by a basis set of magnetic dipoles, and the total E-field at the surface of interest (Aₜₒₜ) caused by the basis set, receiving location information of the electromagnetic coil, aligning the boundary model with the electromagnetic coil map, determining the incident E-field (E_{inc}) of the electromagnetic coil at the surface of interest, determining basis function coefficients (m) that A_{inc} to Eᵢₙ_{^{c}} at the surface of interest, determining an approximation (E^{d}ₜₒₜ) of the total E-field of the electromagnetic coil at the surface of interest, wherein: (E^{d}ₜₒₜ)=Aₜₒₜmî, and outputting the approximation.

### CROSS-REFERENCE TO RELATED APPLICATIONS

This application is based on, claims priority to, and incorporates herein by reference in its entirety U.S. Ser. No. 63/150,549 filed Feb. 17, 2021, and entitled “SYSTEM AND METHOD FOR FAST EVALUATION OF TMS INDUCED ELECTRIC FIELDS USING AN INDIVIDUALIZED MAGNETIC STIMULATION PROFILE.”

### STATEMENT REGARDING FEDERALLY SPONSORED RESEARCH

This invention was made with government support under 1R01MH128421-01, 5R01MH111829-04, and 5P41EB030006-02 awarded by the National Institutes of Health. The government has certain rights in the invention.

### FIELD

The present disclosure relates generally to neuromodulator systems and, more particularly, to systems and methods for profiling a subject and neuronavigation that enable real-time determination of electric fields stimulated by an electromagnetic coil and that enable visualization of induced electric fields in the subject resulting in faster and more accurate placement of the coil about the subject.

### BACKGROUND

Transcranial magnetic stimulation (TMS) is a noninvasive neuromodulation technique used for neuroscience and clinical applications, including diagnostic (e.g., motor system biomarkers, pre-surgical mapping) and therapeutic (e.g., major depression disorder, obsessive compulsive disorder, migraines). TMS uses magnetic fields to stimulate nerve cells in the brain of the subject. The magnetic fields are generated by an electromagnetic coil that is placed over the scalp of the subject to induce electric currents in the underlying brain tissue. The magnetic fields are generated by electric pulses applied to and flowing though the electromagnetic coil. The magnetic fields pass through the skull and into the brain. The position of the coil over the scalp of the subject is selected to focus on and target a specific area or site of the brain for stimulation. The target region of the brain is usually determined by the structure or function of the brain estimated using neuroimaging techniques such as functional magnetic resonance imaging (fMRI). The brain target is typically selected on the surface of the brain, which is not visible in the real space. The goal of TMS is to generate sufficiently strong magnetic fields in a specific region of the brain of the patient to stimulate the appropriate brain network based on the diagnostic or therapeutic application. It may be desirable to place the TMS coil in a position selected so that, for example, the target region in the brain has the strongest possible electric field.

The definition and selection of the coil position on the surface of the skull to optimally engage the desired brain target is critical for all applications, but certainly essential for diagnostic and therapeutic clinical uses. In addition, accurate localization of a brain target is critical to reaching optimal treatment response in TMS. Typically, the coil may be positioned over the head based on external landmarks and measurements. Given that TMS is noninvasive, TMS practitioners have traditionally relied on either skull fiducial markers or stereotactic neuronavigation systems that assume the point with the shortest distance from the brain target to the skull is where the coil should be located. However, these techniques do not take into account the impact of tissues between the coil and the brain on the distribution of the magnetic and induced electric fields. This can lead to inaccuracies in positioning the coil and targeting the brain area. Because of complex brain structure, the point on the scalp that is closest to the brain target may not be the optimal position. Moreover, the electric field (E-field) induced in the brain not only depends on the position of the coil but also depends on the orientation of the coil. In addition, the induced E-field is influenced by tissue conductivity boundaries.

Neuronavigation systems and electric field simulation are techniques that are currently used in clinical settings to improve the localization of the brain target in TMS and overcome problems of positioning based on external landmarks. Neuronavigation is a computer assisted system that is used in TMS to ensure that the relative position of the coil and the head in real space matches the position in image space. Neuronavigation technology visualizes the patient's brain based on imaging data, for example, magnetic resonance imaging (MRI) data), in order to navigate and position the coil to target the desired brain structure or region. In addition, neuronavigation systems may be configured to track and monitor the position of the coil (e.g., using an infrared camera(s) in combination with the imaging data) on a reconstruction of the patient's head or brain during the duration of the TMS stimulation session. Neuronavigation systems can track the position of the TMS coil relative to a target or relative to an anatomical area of interest. Some neuronavigation systems may comprise a robotic element (e.g. arm) to position the TMS coil and may determine TMS coil position accordingly, such as by stepper motor feedback or other suitable system or method.

Typically, software is used for E-field simulation to estimate the E-field for a specific coil position and orientation. In some instances, E-field simulation may make use of an approximate model of the head. However, current E-field simulation approaches usually take several minutes to compute the E-field for a specific coil position. If that coil position is not optimal, then a different position needs to be examined. Because of the long estimation time, only a few positions are typically examined in practice, making the selected coil position sub-optimal. In addition, current E-simulation tools are not practical in clinical setting because they are too slow to be integrated with neuronavigation systems to respond to any adjustment of coil position by clinicians in real time. In some cases, faster simulation results may be provided using rough approximations of the head (e.g. a spherical model), but these do not provide anatomically realistic or accurate modelling of the E-field. Moreover, clinicians are not fully informed by neuronavigation systems about the stimulated brain site relative to different coil positions.

It would be desirable to provide a system and method that enables accurate real-time determination of the E-field induced by a TMS coil, and enables, for example, accurate real-time graphical rendering of E-fields stimulated by an electromagnetic coil at multiple position per second.

### SUMMARY

In accordance with some embodiments of the disclosed subject matter, systems, methods, and media for E-field determination are provided.

In accordance with some embodiments of the disclosed subject matter, an E-field determination system for an electromagnetic coil positioned about a subject having one or more conductivity boundaries, is provided, the system comprising a memory configured to store therein: a predetermined electromagnetic coil E-field map; a predetermined boundary model associated with the subject, wherein the boundary model comprises a model of a surface of a first conductivity boundary of the subject; and a predetermined Magnetic Stimulation Profile (MSP) associated with the subject, wherein the MSP comprises: the incident E-field at a first surface of interest (A_{inc}) caused by a basis set of magnetic dipoles; and the total E-field at the first surface of interest (Aₜₒₜ) caused by the basis set of magnetic dipoles; and a processor communicatively coupled with the memory and configured to: (a) receive a location information of the electromagnetic coil; (b) align, based on the received location information, the predetermined boundary model with the predetermined electromagnetic coil E-field map; (c) determine the incident E-field (E_{inc}) of the electromagnetic coil at the first surface of interest based on the aligned predetermined electromagnetic coil E-field map and predetermined boundary model; (d) determine basis function coefficients (m) that match the incident E-field (A_{inc}) of the basis set of magnetic dipoles to the determined incident E-field of the electromagnetic coil (E_{inc}) at the first surface of interest; (e) determine an approximation (Eₜₒₜ^{d}) of the total E-field of the electromagnetic coil at the first surface of interest, wherein: Eₜₒₜ^{d}=Aₜₒₜ\hat{m}; and (f) output the approximation (Eₜₒₜ^{d}) of the total E-field of the electromagnetic coil at the first surface of interest.

In some embodiments, the system is configured to repeat (b) to (e) for changing location information at least five times in a second.

In some embodiments, the predetermined electromagnetic coil E-field map comprises an interpolating function (F_{inc}^{g}); and determining the incident E-field (E_{inc}) of the electromagnetic coil at the first surface of interest comprises using the interpolating function (F_{inc}^{g}) on the first surface of interest.

In some embodiments, aligning the predetermined boundary model with the predetermined electromagnetic coil E-field map comprises performing transform T_{c}⁻¹ on the boundary model, wherein:

$${T}_{c} = [ \begin{matrix}{R}_{c} & {T}_{0} \\ 0 & 1\end{matrix} ] ,$$

wherein R_{c} comprises the three-dimensional rotation matrix and T₀ comprises the translation vector from a previous location of the electromagnetic coil to a current location of the electromagnetic coil according to the received location information.

In some embodiments, the basis function coefficients (\hat{m}) are determined by: \hat{m}=WE_{inc}, wherein

$$W = {( {A}_{i n c}^{T} {A}_{i n c} + {λ}^{2} I )}^{- 1} {A}_{i n c}^{T} ,$$

wherein λ is a regularization parameter.

In some embodiments, the surface of interest comprises a conductivity boundary.

In some embodiments, the basis set of magnetic dipoles comprises a plurality of sets of three orthogonal magnetic dipoles located on a second surface around the model of the surface of the first conductivity boundary.

In some embodiments, the total E-field at the first surface of interest (Aₜₒₜ) caused by the basis set of magnetic dipoles is determined by the summation of the incident E-field at the first surface of interest (A_{inc}) caused by the basis set of magnetic dipoles and a secondary E-field at the first surface of interest (A_{S}) caused by a charge accumulation.

In some embodiments, the secondary E-field at the first surface of interest (A_{S}) is determined according to a Boundary Element Method utilizing Fast Multilevel Multipole (BEM-FMM).

In some embodiments, the MSP comprises: the incident E-field at a plurality of surfaces of interest (Ā_{inc}) caused by the basis set of magnetic dipoles, wherein A_{inc} is a subset of Ā_{inc}; and the total E-field at a plurality of surfaces of interest (Āₜₒₜ) caused by the basis set of magnetic dipoles, wherein Aₜₒₜ is a subset of Āₜₒₜ.

In accordance with some embodiments of the disclosed subject matter, a method for E-field determination for an electromagnetic coil positioned about a subject having one or more conductivity boundaries, is provided, the method comprising: (a) retrieving, from a memory: a predetermined electromagnetic coil E-field map; a predetermined boundary model associated with the subject, wherein the boundary model comprises a model of a surface of a first conductivity boundary of the subject; and a predetermined Magnetic Stimulation Profile (MSP) associated with the subject, wherein the MSP comprises: the incident E-field at a first surface of interest (A_{inc}) caused by a basis set of magnetic dipoles; and the total E-field at the first surface of interest (Aₜₒₜ) caused by the basis set of magnetic dipoles; (b) receiving, using a processor, a location information of the electromagnetic coil; (c) aligning, using the processor and based on the received location information, the predetermined boundary model with the predetermined electromagnetic coil E-field map; (d) determining, using the processor, the incident E-field (E_{inc}) of the electromagnetic coil at the first surface of interest based on the aligned predetermined electromagnetic coil E-field map and predetermined boundary model; (e) determining, using the processor, basis function coefficients (\hat{m}) that match the predetermined incident E-field at the first surface of interest (A_{inc}) to the determined incident E-field of the electromagnetic coil (E_{inc}) at the first surface of interest; (f) determining, using the processor, an approximation (Eₜₒₜ^{d}) of the total E-field of the electromagnetic coil at the first surface of interest, wherein: Eₜₒₜ^{d}=Aₜₒₜ\hat{m}; and (f) outputting the approximation (Eₜₒₜ^{d}) of the total E-field of the electromagnetic coil at the first surface of interest.

In some embodiments, the method further comprises repeating (c) to (f) for changing location information at least five times in a second.

In some embodiments, the predetermined electromagnetic coil E-field map comprises an interpolating function (F_{inc}^{g}); and determining the incident E-field (E_{inc}) of the electromagnetic coil at the first surface of interest comprises using the interpolating function (F_{inc}^{g}) on the first surface of interest.

In some embodiments, aligning the predetermined boundary model with the predetermined electromagnetic coil E-field map comprises performing transform T_{c}⁻¹ on the boundary model, wherein:

$${T}_{c} = [ \begin{matrix}{R}_{c} & {T}_{0} \\ 0 & 1\end{matrix} ] ,$$

wherein R_{c} comprises the three-dimensional rotation matrix and T comprises the translation vector from a previous location of the electromagnetic coil to a current location of the electromagnetic coil according to the received location information.

In some embodiments, determining the basis function coefficients (\hat{m}) comprises: \hat{m}=WE_{inc}, wherein W=(A_{inc}^{T}A_{inc}+λ²I)⁻¹A_{inc}^{T}, wherein λ is a regularization parameter.

In some embodiments, the basis set of magnetic dipoles comprises a plurality of sets of three orthogonal magnetic dipoles located on a second surface around the model of the surface of the first conductivity boundary.

In some embodiments, the predetermined total E-field at the first surface of interest (Aₜₒₜ) caused by the basis set of magnetic dipoles is determined by the summation of the predetermined incident E-field at the first surface of interest (A_{inc}) caused by the basis set of magnetic dipoles and a predetermined secondary E-field at the first surface of interest (A_{S}) caused by a charge accumulation.

In some embodiments, the predetermined secondary E-field at the first surface of interest (A_{S}) is determined according to a Boundary Element Method utilizing Fast Multilevel Multipole (BEM-FMM).

In some embodiments, the MSP comprises: the incident E-field at a plurality of surfaces of interest (Ā_{inc}) caused by the basis set of magnetic dipoles, wherein A_{inc} is a subset of Ā_{inc}; and the total E-field at a plurality of surfaces of interest (Āₜₒₜ) caused by the basis set of magnetic dipoles, wherein Aₜₒₜ is a subset of Āₜₒₜ.

In accordance with some embodiments of the disclosed subject matter, a system for positioning an electromagnetic coil about a subject, is provided, the system comprising: a memory having stored therein: a predetermined electromagnetic coil E-field map; a predetermined boundary model associated with the subject, wherein the boundary model comprises a model of a surface of a first conductivity boundary of the subject: a predetermined Magnetic Stimulation Profile (MSP) associated with the subject; a processor communicatively coupled with the memory and configured to: (a) receive a location information of the electromagnetic coil; (b) align, based on the received location information, the predetermined boundary model with the predetermined electromagnetic coil E-field map; (c) determine an incident E-field (E_{inc}) of the electromagnetic coil at the first surface of interest based on the aligned predetermined electromagnetic coil E-field map and predetermined boundary model; (d) determine an approximation (Eₜₒₜ^{d}) of the total E-field of the electromagnetic coil at the first surface using the MSP and the incident E-field; and (e) generate a report indicating (Eₜₒₜ^{d}) of the total E-field of the electromagnetic coil at the first surface of interest.

### BRIEF DESCRIPTION OF THE DRAWINGS

The present disclosure will hereafter be described with reference to the accompanying drawings, wherein like reference numerals denote like elements.

FIG. 1 is a block diagram of an example transcranial magnetic stimulation (TMS) system in accordance with an embodiment;

FIG. 2 is a block diagram of a system for integrated electric field simulation and neuronavigation in accordance with an embodiment;

FIG. 3 is a block diagram of an example computer system in accordance with an embodiment;

FIG. 4 is a block diagram of a workflow of the system of FIG. 2 for generating a graphical rendering of an electric field simulation in real-time in accordance with an embodiment;

FIG. 5 illustrates a method for generating a graphical rendering of an electric field simulation in accordance with an embodiment;

FIG. 6 illustrates a determined E-field map of an electromagnetic coil in accordance with an embodiment;

FIG. 7 illustrates a method for generating a boundary model in accordance with an embodiment;

FIG. 8 illustrates the results of a method for generating a boundary model in accordance with an embodiment;

FIG. 9 illustrates a cross-section of the calculated total E-field in accordance with an embodiment;

FIG. 10 illustrates a dipole basis set in accordance with an embodiment;

FIG. 11 illustrates a method for predetermining a Magnetic Stimulation Profile in accordance with an embodiment;

FIG. 12 illustrates a method for predetermining inputs to an E-field approximation process in accordance with an embodiment;

FIG. 13 illustrates a method for approximating total E-field in accordance with an embodiment;

FIG. 14 illustrates approximated and numerically accurate incident E-fields caused by a moved electromagnetic coil in accordance with an embodiment;

FIG. 15 illustrates determined dipole basis set coefficients, incident E-field, and approximated total E-field caused by an electromagnetic coil in accordance with an embodiment;

### DETAILED DESCRIPTION

The present disclosure describes systems and methods for integrating electric field (E-field) simulation (also referred to herein as E-field “determination”) with neuronavigation to improve targeting of brain regions for neuromodulation techniques such as, for example, transcranial magnetic stimulation (TMS). In some embodiments, integrated E-field simulation and neuronavigation system(s) according to the present disclosure may be used to generate a graphical rendering of electric fields for a coil position which, for example, enables the visualization of the electric field in the brain in real time after adjusting the coil position. Correct position and orientation of the coil can be, for example, important for treatment planning for a neuromodulation technique such as TMS and can improve the accuracy of diagnostic applications. Advantageously, the E-field determination may be performed using a pre-calculated (predetermined) Magnetic Stimulation Profile (MSP) unique to each subject which allows for ultra-fast computation and rendering of E-fields that are generated (induced) when the brain is stimulated using an electromagnetic coil (e.g., a TMS coil). In addition, the disclosed systems and methods for integrating E-field simulation and neuronavigation can also advantageously be used to provide real time (e.g. several times a second) determination of the induced E-field as an electromagnetic coil is moved about the subject, providing real time feedback to the neuronavigation system and/or an operator of the electromagnetic coil to allow accurate targeting of stimulation.

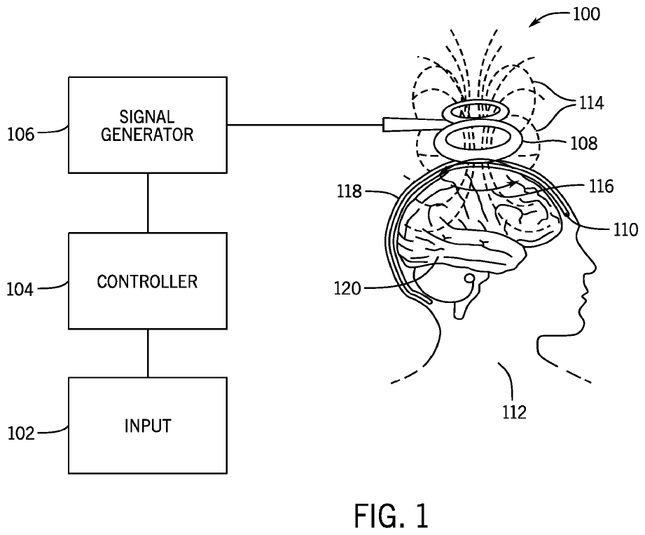

FIG. 1 is a block diagram of an example transcranial magnetic stimulation (TMS) system in accordance with an embodiment. A TMS system 100 may include an input 102, a controller 104, a signal generator 106 and an electromagnetic coil 108. The controller 104 is in communication with the signal generator 106 and is configured to direct the signal generator 106 to provide various signals to the coil 108. In some implementations, the controller 104 may be any general-purpose computing system or device, such as a personal computer, workstation, cellular phone, smartphone, laptop, tablet, or the like. As such, the controller 104 may include any suitable hardware and components designed or capable of carrying out a variety of processing and control tasks, for example including steps for optimizing and directing the signal generator 106 to provide various signals to the coil 108. For example, the controller 104 may include a programmable processor or combination of programmable processors, such as central processing units (CPUs), graphics processing units (GPUs), and the like. In some embodiments, the TMS system 100 may comprise a multichannel TMS system, having multiple coils or coil arrays driven by multichannel stimulators. In some implementations, the controller 104 may be configured to execute instructions stored in a non-transitory computer readable-media. In this regard, the controller 104 may be any device or system designed to integrate a variety of software, hardware, capabilities and functionalities. Alternatively, and by way of particular configurations and programming, the controller 104 may be a special-purpose system or device. For instance, such special-purpose system or device may include one or more dedicated processing units or modules that may be configured (e.g., hardwired, or pre-programmed) to carry out steps, in accordance with aspects of the present disclosure.

The electromagnetic coil 108 is positioned about (e.g. proximate to and over) the head, for example, the scalp 118, of a subject 112. The electromagnetic coil 108 may be insulated using known methods and materials. In some embodiments, the coil 108 may be positioned and held in place over the scalp 118 by an operator or using a mechanical arm (not shown). The position of the coil 108 over the scalp 118 is selected to target and stimulate a specific area of the brain (e.g., a region, site or target in the brain). Accordingly, the coil 108 may be positioned over the region to be stimulated in the brain. Signal generator 106 is configured to generate and deliver electrical signals (e.g., electric current or voltage signals) to the coil 108. The electric signal delivered from the signal generator 106 and flowing through the coil 108 generates a time-varying magnetic field 114. The time-varying magnetic field 114 (e.g., magnetic pulses) passes through the skull 110 and into the brain 120 of the subject 112 and causes or induces electrical currents 116 and electric fields that stimulate nerve cells in the targeted brain region. Different coil types may be used for coil 108 to elicit different magnetic field patterns. The strength and distribution of the time-varying magnetic fields 114 may be dependent on both the geometry (e.g. number of coils, coil size, etc.) and the amount of current traveling through the coil 108.

The induced electric current and electric field 116 may also be dependent on variables unique to individual subjects such as the geometry and electrical properties of anatomies in and around the brain. For example, the induced electric current and E-field 116 may be influenced by tissue conductivity boundaries, such as the skin/scalp, skull, cerebro-spinal fluid (CSF), grey matter (GM), and white matter (WM). The conductivity may be assumed to be homogeneous within each tissue compartment defined by the boundary surfaces.

In some embodiments, the signals generated by the signal generator 106 and provided to the coil 108 may be in the form of a pulse sequence having a plurality of pulses. In some embodiments, the pulse frequency may be up to 10 kHz. The power, amplitude, duration, shape, and frequency of the pulses may be selected to achieve a desired level of or depth of stimulation, as well as to optimize heat or magnetic forces induced in the coil 108. An operator may select the specific type and characteristics of the electric pulses to be generated by the signal generator 106 using an input 102 coupled to the controller 104. The input 103 can be, for example, a keyboard, a mouse, a touch screen, etc. While the following description will be discussed in referenced to a TMS system and a TMS coil, it should be understood that the systems and methods described herein may be used with other types of non-invasive neuromodulation systems or other types of electromagnetic stimulation, and the systems and methods described herein may be used on subjects having one or more conductivity boundaries and not limited to the head of an individual human.

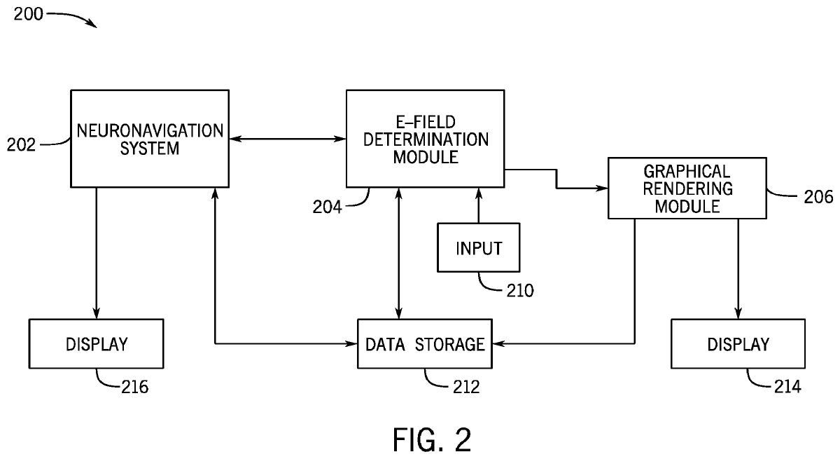

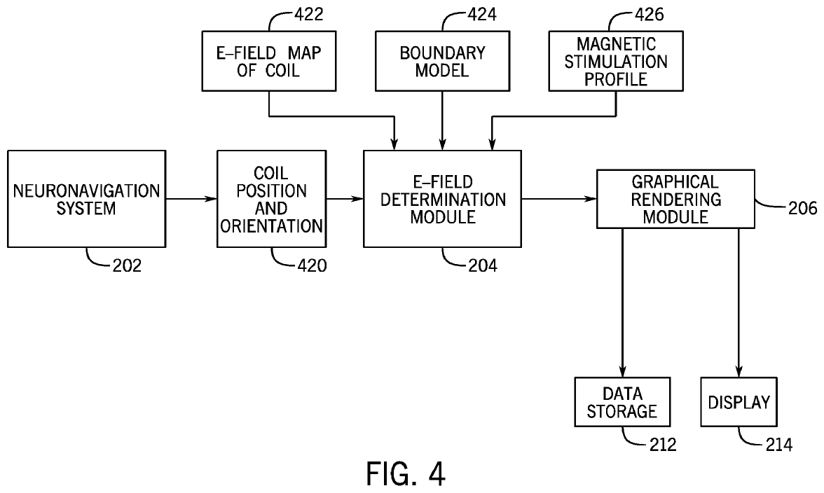

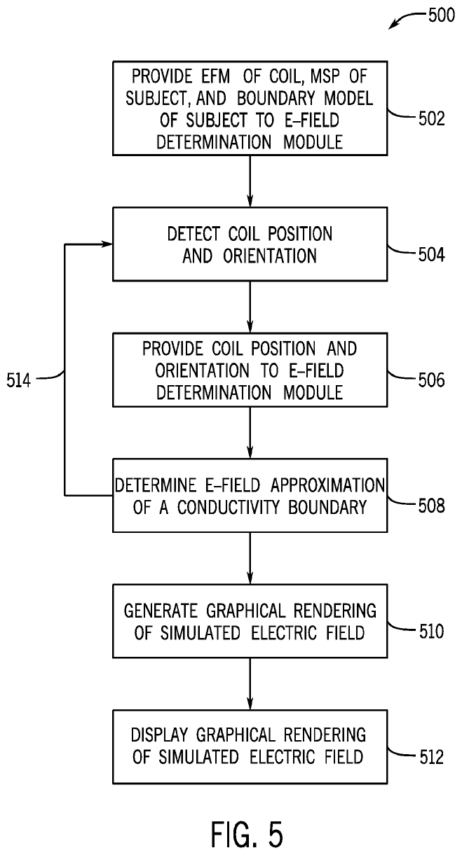

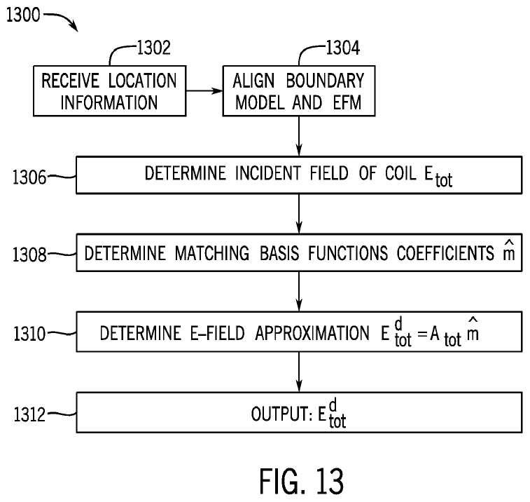

FIG. 2 is a block diagram of an integrated TMS system 200 for integrated electric field simulation and neuronavigation in accordance with some embodiments. The integrated TMS system 200 includes a neuronavigation system 202, an electric field (E-field) determination unit 204, and a graphical rendering module 206. The neuronavigation system 202 may be configured to, for example, visualize a subject's brain based on imaging data, for example, magnetic resonance imaging (MRI) data, and to track, monitor, and/or visualize the position of a coil (e.g., a TMS coil) about the subject's head or brain. In some embodiments, the neuronavigation system 202 can track the position of the TMS coil relative to a target or relative to an anatomical area of interest. The neuronavigation system 202 may be any known neuronavigation system in the art. Neuronavigation system 202 may be coupled to a display 216 to display images of a subject's head and brain, tracking of a coil used for TMS, and other information associated with the subject being treated with TMS. The neuronavigation system 202 may also be coupled to data storage 212 from which it may retrieve data or may provide information to for storage. The neuronavigation system 202 may be coupled to and in signal communication with the E-field determination module 204. The neuronavigation system 202 and the E-field determination module 204 may be configured to exchange data such as, for example, the neuronavigation system 202 may provide a current coil position and orientation to the E-field determination module 204 for analysis as discussed further below with respect to FIGS. 4 and 5, and the E-field determination module 204 may provide an E-field approximation, for example to the neuronavigation system 202, as discussed further below with respect to FIG. 13.

The E-field determination module 204 is configured to generate an approximation (also referred to as an estimation or simulation) of the E-fields that may be generated by stimulation of a subject using a TMS coil. The subject may comprise a head, including brain. As discussed further below, the E-field determination module 204 may be configured to generate an E-field simulation based on a plurality of inputs 210 including, but not limited to, a position and orientation of a TMS coil and an electric field map of the TMS coil. In some embodiments, the inputs 210 may comprise neuroimaging data (such as multimodal neuroimaging) associated with a subject being treated with a TMS system, a pre-calculated (also referred to as predetermined) boundary model associated with the subject as discussed further below, and/or a pre-calculated (also referred to as predetermined) Magnetic Stimulation Profile (MSP) associated with the subject as discussed further below. The input 210 may be provided by an operator (e.g., by using an input such as a keyboard, a mouse, a touch screen, etc.) or may be retrieved from data storage (or memory) 212.

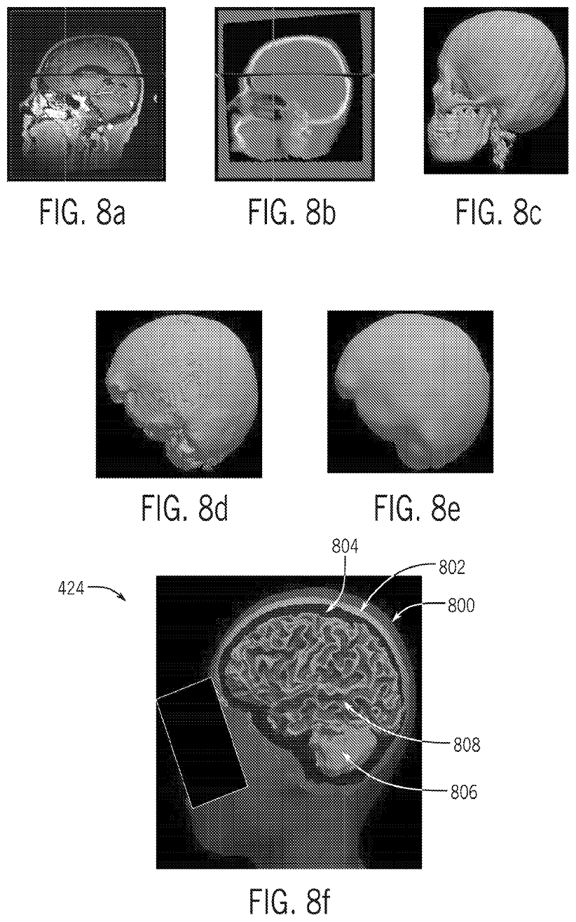

The E-field determination module 204 may comprise any suitable hardware and/or software. The E-field determination module 204 may comprise the same or different hardware as the neuronavigation system 202. For example, the E-field determination module 204 may comprise a computer programmed to perform specific methods. The E-field determination module 204 may be coupled to a graphical rendering module 206 and/or data storage 212. In some embodiments, the neuronavigation system 202 provides a current position and orientation of a TMS coil to the E-field determination module 204 which estimates the E-fields for the current position and orientation. The estimated E-fields can then be provided to and used by the graphical rendering module 206 to generate a graphical rendering of the estimated E-fields. Known methods may be used to generate the graphical rendering of the estimated E-fields. In an embodiment, the E-field determination module 204 and the graphical rendering module 206 may be implemented on a graphics processing unit (GPU) (e.g., GPU 808 shown in FIG. 8) of a computer system. As discussed below with respect to FIGS. 4 and 5, the E-field determination module 204 can enable the visualization of the electric field in the head (including brain) in real time for a moving coil position. Graphical rendering module 206 may also be coupled to a display 214 which may be used to display the graphical rendering. In some embodiments, the display 214 and the display 216 may be separate displays or may be the same display.

Conventional E-field simulation approaches are typically too slow for a clinical setting and are limited to determining the E-field for a specific brain region. Advantageously, in the present disclosure, the E-field simulation may be performed using the E-field determination module 204 using a MSP that allows for ultra-fast computation and rendering of E-fields that may be generated when the brain is stimulated using an electromagnetic coil (e.g., a TMS coil). In addition, the E-field determination module 204 can be configured to generate a global electric field estimation for a neuromodulation technique such as TMS. Accordingly, the E-field determination module 204 may be configured to estimate the E-field for the whole-brain of a subject for any coil position and orientation, or for one or more conductive boundaries of the subject. For example, in some embodiments, the E-field determination module 204 may be configured to determine the E-field of a region of interest, which may comprise a conductivity boundary or other surface such as a planar two-dimensional (2D) slice or other part of the 3D brain volume, at a rate of at least 5 times per second (5 Hz), and in some embodiments at least 10-15 times per second (10-15 Hz), for a particular coil position and orientation. In some embodiments, the E-field determination module 204 generates an estimated E-field for a selected region of the brain of the subject.

In some embodiments, the E-field determination module 204 may generate an estimation of the E-fields for a coil orientation and position based on inputs including an electric field map (EFM) for the type of electromagnetic coil being used in the TMS system and neuroimaging data associated with the subject and/or a boundary model of the subject. Electric field maps can be different for different types of TMS coils. The multimodal neuroimaging data may include, for example, anisotropic conductivity of the brain tissue of the subject and tissue segmentation obtained using anatomical MRI. Anisotropic conductivity of brain tissue may be determined using, for example, diffusion magnetic resonance imaging (MRI). A boundary model is described further below.

Various aspects of the integrated TMS system 200 may be implemented with one or more computer systems. For example, the neuronavigation system 202 may comprise a computer system as described above, and the E-field determination module 204 may comprise a computer system. In some embodiments, the neuronavigation system 202 and E-field determination module 204 may be implemented on the same computer system. In some embodiments, the neuronavigation system 202 and E-field determination module 204 may be implemented on different computer systems.

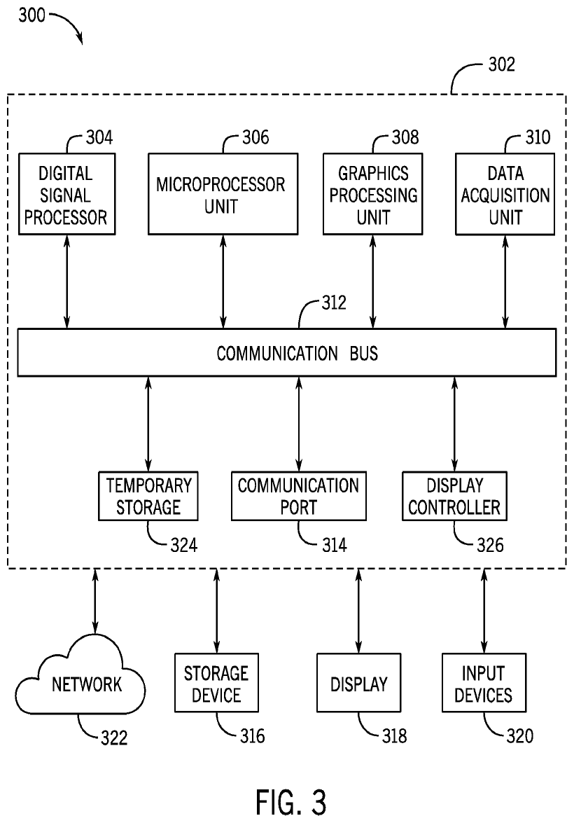

FIG. 3 is a block diagram of an example computer system in accordance with some embodiments. Computer system 300 may be used to implement the systems and methods described herein. In some embodiments, the computer system 300 may be a workstation, a notebook computer, a tablet device, a mobile device, a multimedia device, a network server, a mainframe, one or more controllers, one or more microcontrollers, or any other general-purpose or application-specific computing device. The computer system 300 may operate autonomously or semi-autonomously, or may read executable software instructions from the memory or storage device 316 or a computer-readable medium (e.g., a hard drive, a CD-ROM, flash memory), or may receive instructions via the input device 322 from a user, via the network 322, or any other source logically connected to a computer or device, such as another networked computer or server. Thus, in some embodiments, the computer system 300 can also include any suitable device for reading computer-readable storage media.

Data, such as data acquired with an imaging system (e.g., a CT imaging system, a magnetic resonance imaging (MRI) system, etc.), MSP, EFM, boundary model, or the like, may be provided to the computer system 300 from a data storage device 316, and these data are received in a processing unit 302. In some embodiment, the processing unit 302 includes one or more processors. For example, the processing unit 302 may include one or more of a digital signal processor (DSP) 304, a microprocessor unit (MPU) 306, and/or a graphics processing unit (GPU) 308. The processing unit 302 also includes a data acquisition unit 310 that is configured to electronically receive data to be processed. The DSP 304, MPU 306, GPU 308, and data acquisition unit 310 may be coupled to a communication bus 312. The communication bus 312 may comprise, for example, a group of wires, routing on a semiconductor die, hardware used for switching data between the peripherals or between any components in the processing unit 302, and the like.

The processing unit 302 may also include a communication port 314 in electronic communication with other devices, which may include a storage device 316, a display 318, and one or more input devices 320. Examples of an input device 320 include, but are not limited to, a keyboard, a mouse, and a touch screen through which a user can provide an input. The storage device 316 may be configured to store data, which may include data such as, for example, electric field maps of different types of electromagnetic coils, multimodal neuroimaging data, imaging data, MSP of a subject, boundary model of a subject, etc., whether these data are provided to, retrieved by, or processed by, the processing unit 302. The display 318 may be used to display images and other information, such as magnetic resonance images, E-field information, patient health data, and so on.

The processing unit 302 can also be in electronic communication with a network 322 to transmit and receive data and other information. The communication port 314 can also be coupled to the processing unit 302 through a switched central resource, for example the communication bus 312. The processing unit can also include temporary storage 324 and a display controller 326. The temporary storage 324 is configured to store temporary information. For example, the temporary storage 324 can be a random access memory.

The memories described herein may be of any suitable type, such as volatile or non-volatile, local or remote (cloud, networked, etc.), and may comprise multiple memories of any suitable combination. The memory may be configured to be large enough to store relevant information, such as the boundary model, MSP, EFM, and/or program code configured to perform the methods described herein.

Computer-executable instructions for real-time E-field estimation, EFM determination, MSP determination, boundary model determination, real-time graphical rendering of electric fields stimulated by an electromagnetic coil, and the like according to the methods described herein may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by internet or other computer network form of access.

As mentioned above, the integrated TMS system 200 (shown in FIG. 2) may be configured to generate graphical renderings of simulated electric fields in a region of interest in real time. FIG. 4 is a block diagram of a workflow of the system of FIG. 2 for generating a graphical rendering of an electric field simulation in real-time in accordance with an embodiment and FIG. 5 illustrates a method 500 for generating a graphical rendering of an electric field simulation in accordance with an embodiment. Referring to FIGS. 4 and 5, at block 502, the EFM 422 of the TMS coil, and a boundary model 424 and MSP 426 associated with the subject are provided as inputs to the E-field determination module 204. At block 504, a neuronavigation system 202 may be used to detect a coil position and orientation 420 for a TMS coil (e.g., coil 108 shown in FIG. 1). At bock 506, the coil position and orientation 420 may be provided to an E-field determination module 204. In some embodiments, providing the various information/inputs to the E-field determination module 204 may comprise storing the information in data storage, such as data storage 212, a memory of the neuronavigation system 202 and/or E-field determination module, and/or other computer system, wherein it is retrievable by the E-field determination module 204, electronically communicating the information, or other suitable method. In some embodiments, the coil position and orientation 420 may be provided 506 by an operator using, for example, an input (e.g., input 210 shown in FIG. 2). In some embodiments, coil position and orientation 420, the EFM 422 of the coil, the MSP 426, and/or the boundary model 424 may be retrieved from data storage (e.g., memory) of, for example, the E-field determination module 204, the neuronavigation system 202, data storage 212, and/or other computer system.

At block 508, the E-field determination module 204 generates an estimation or prediction of electric fields that may be generated, for example on a surface of interest, when the subject's brain is stimulated using the coil. As discussed above, the determination of the estimated electric fields may be based on the inputs including the detected coil position and orientation 420, the EFM 422 of the coil, and the MSP 426 and boundary model 424 associated with the subject. In some embodiments, the E-field determination module 204 generates an estimated E-field for the whole brain of the subject. In some embodiments, the E-field determination module 204 generates an estimated E-field for a selected region of the brain of the subject. At block 510, a graphical rendering module 206 is used to generate a graphical rendering of the simulated E-fields. The graphical rendering may be generated using rendering methods known in the art. In an embodiment, the E-field determination module 204 and the graphical rendering module 206 may be implemented on a graphics processing unit (GPU) (e.g., GPU 808 shown in FIG. 8) of a computer system. At block 512, the graphical rendering may be provided to and displayed on a display 214. The graphical rendering may be stored in data storage (e.g, memory) 212 of the integrated TMS system 200, data storage (e.g., memory) of the E-field determination module 204 and/or the neuronavigation system 202, or other computer system.

Advantageously, the E-field determination module 204 and graphics rendering module 206 may be used to visualize the electric field in real-time after an operator adjusts the position of the coil (or after the position is adjusted automatically by the neuronavigation system 202). Once the coil position and orientation are changed by an operator (and detected 504 by the neuronavigation system 202), the estimated E-field may also be adjusted in real-time to provide information about the actual stimulation of the brain with the new coil position and orientation. In some embodiments, integrated TMS system 200 may be configured to repeat 514 the steps of position detection 504 through E-field determination 508 several times a second, for example at least 5 times a second, and in some embodiments at least 10 or 15 times a second. Advantageously, the E-field determination module 204 may be configured to estimate the E-field (e.g., E-field of a whole brain or a region of interest) within 200 ms or less for a particular coil position and orientation, and in some embodiments within 100 ms or less for a particular coil position and orientation.

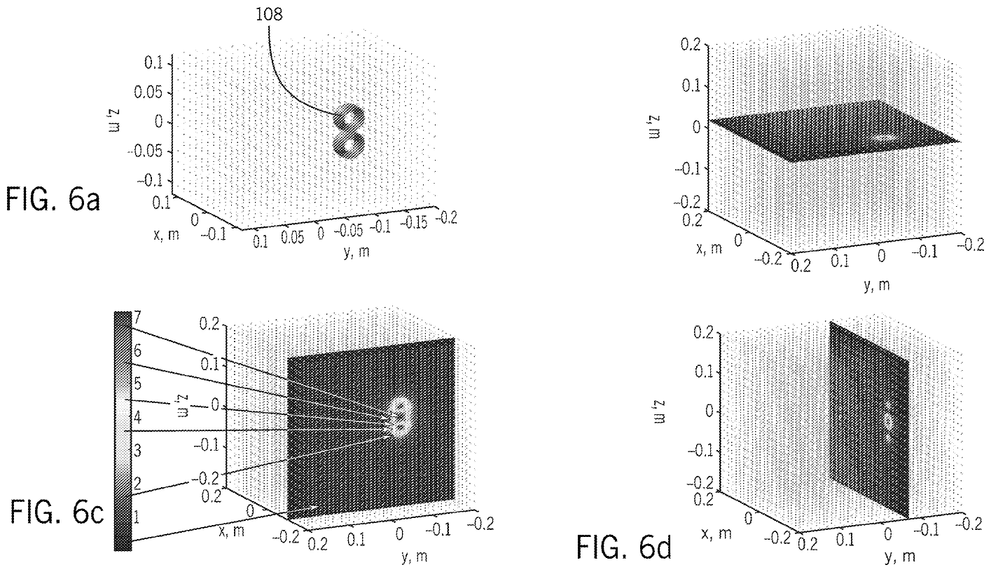

An electric field map (EFM) for an electromagnetic coil comprises a characterization (or model) the incident E-field caused by the coil. In some embodiments, EFM may comprise a three-dimensional (3D) characterization of coil's incident E-field. The EFM may be pre-calculated (predetermined) in any suitable manner. Referring to FIG. 6, in some embodiments the EFM may be pre-calculated using a grid interpolation method. For example, a model of the coil may be positioned inside dense 3D spatial grid as illustrated in FIG. 6(a). In an embodiment, the grid may comprise a cubic grid with millions of points, for example 9 million points. The incident E-field of the coil may be calculated on all the grid points from which an interpolating function (F_{inc}^{g}) can be determined using any suitable method. The incident E-field of the coil on grid points may be determined using any suitable method. In some embodiments, the incident E-field of the coil may be determined by calculating the electric potentials at each point within the cubic grid using a Fast Multilevel Multipole method. In an embodiment, interpolation function determination may be accomplished by trilinear interpolation based on the incident E-field values at eight corners of each sub-cube within the full grid. For example, standard mathematical software programs may be used to define the interpolating function (F_{inc}^{g}). FIGS. 6(b)-6(d) show the calculated incident E-field magnitude of the coil at different cross-sectional planes.

Using the approach described above, E-field maps (EFM) of any number of electromagnetic coils-whether different manufacturers, coil configurations, size, etc.—may be determined and stored (e.g., in data storage 212) for later use by the E-field determination module 204. In this way, a library of coil EFM's can be provided. Advantageously, each coil EFM only need be calculated once, and can be calculated prior to use by the E-field determination module 204.

The boundary model 424 may comprise a characterization (or model) of the conductivity boundaries within a subject. In some embodiments, the boundary model 424 may comprise conductivity compartments that are nested and non-intersecting, and the conductivity boundaries may be closed manifolds that are topologically equivalent to a sphere. In some embodiments, the boundary model 424 may comprise a volume conductor model, for example of the subject's head. The conductivity boundaries may be defined by surfaces separating different conductivity regions within the subject, and determining the boundary model 424 may comprise generating surface meshes using neuroimaging data. Neuroimaging data 424 may be, for example, neuroimaging data of a subject's head and brain (e.g., functional and structural information) acquired using a plurality of different modalities including, for example, MRI (e.g., functional MRI (fMRI)), positron emission tomography (PET), computed tomography (CT), electroencephalography (EEG), magnetoencephalography (MEG), and near-infrared spectroscopy (NIRS). In some embodiments, the neuroimaging data includes anisotropic conductivity of brain tissue of the subject acquired using MRI. In an embodiment, the multimodal neuroimaging data may include combinations of data sets acquired using different modalities.

Conductivity boundaries may comprise, for example, the skin/scalp (e.g., outer surface of scalp), skull (e.g., boundary between skull and skin), cerebro-spinal fluid (e.g., boundary between CSF volume and skull volume), grey matter (e.g., boundary between GM volume and CSF volume), and white matter (e.g., boundary between WM volume and GM volume). Existing tools can be used to perform brain segmentation and surface analysis to create a model of the intracranial region. To produce accurate surfaces of the extracranial conductivity compartments (skull, skin/scalp) with rigorously defined geometric and topological properties, the method described below may be used.

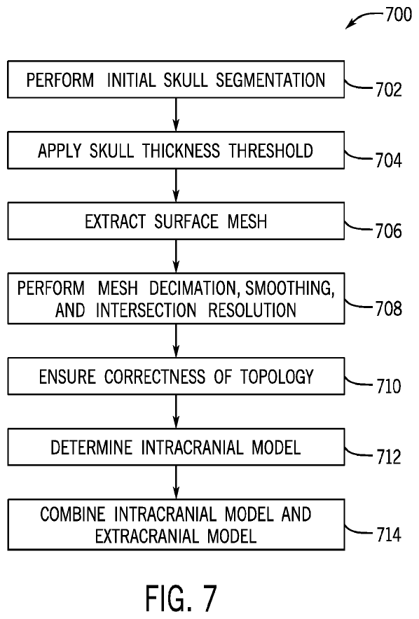

FIG. 7 illustrates a boundary model generation process 700 for generating a boundary model 424, and FIG. 8 illustrates the results at various steps of the method 700. The boundary model generation process 700 may be performed by a computer system, for example using E-field determination module 204 and/or the neuronavigation system 202, or other computer system. To create an extracranial model, the method 700 may start with any suitable neuroimaging data, for example an MRI scan of the subject as shown in FIG. 8(a). Referring to FIGS. 8(b)-8(c), initial skull segmentation 702 may be accomplished using any known method, such as a pseudo-CT, to determine the “raw” skull surface. Referring to FIG. 8(d), a skull thickness threshold may be applied 704 to remove the cheekbones and jaw, which do not have a practical impact on the coil-induced intracranial E-fields. A surface mesh may then be extracted 706 from the simplified skull volume. In some embodiments, surface extraction 706 may comprise using two triangles to represent each (square) face separating voxel classified as different tissues. Other known methods may alternatively be used for surface extraction 706. At block 708, mesh decimation, smoothing, and intersection resolution processes may be performed 708 with fixing of holes, resulting in a skull model as illustrated by FIG. 8(e). In some embodiments, when a thin gap between individual objects is allowed, the shallow intersections between nearby objects may be resolved via deforming the individual meshes in the contact area along the inner normal directions. In some embodiments, a final topology correction process 710 may be performed on the skull and CSF surfaces, which may verify topology correctness and/or perform topology correction.

The boundary model generation process 700 may further comprise generating the intracranial model 712, as described above. The generation of the intracranial model 712 may occur separately from the creation of the extracranial model. The generation of the intracranial model 712 may occur before, after, or during any of the other steps of method 700 described above. The model generation process 700 may further comprise combining the intracranial model with the extracranial model to create a boundary model 424. FIG. 8(f) illustrates an exemplary boundary model 424 of a subject's head which characterizes the various compartments and surfaces, include the skin/scalp 800, skull 802, CSF 804, GM 806, and WM 808. Advantageously, the boundary model 424 can be calculated prior to use by the E-field determination module 204, as described in more detail below.

The total E-field in the subject (induced by the coil) may comprise the combination of the incident E-field from the TMS coil and a secondary E-field caused by charge accumulation at the conductivity boundaries and has the general form E=−∂A/∂t−∇Φ. Conductivity may be assumed to be homogeneous within each tissue compartment defined by the boundary surfaces. For biological tissue with relatively low conductivities, the first term E_{inc}=−∂A/∂t or corresponds to the primary field induced by the coil, and the second term E^{S}=−∇Φ corresponds to the secondary field caused by charge accumulation. The primary and secondary E-fields are coupled through the condition of volumetric quasi-neutrality (∇·J=∇·(σE)=0) and the secondary field is generated by charge accumulation at the conductivity boundaries to render the normal component of the current continuous. The total E-field may be solved by known methods, but previous methods are either too slow or too inaccurate to provide a real-time determination of the total E-field induced by an electromagnetic coil for neuroscience and clinical applications.

In some embodiments, the secondary E-field E^{S} may be solved using a Boundary Element Method utilizing Fast Multilevel Multipole (BEM-FMM). The BEM-FMM method operates directly with electric charges. The surfaces S of all conductivity boundaries may be discretized into N small planar triangles tᵢ, i=1, . . . , N with centers cᵢ, i=1, . . . , N, outer normal vectors of every manifold or non-manifold tissue shell nᵢ, i=1, . . . , N, and triangle areas Aᵢ, i=1, . . . , N. The outer normal vectors may be predefined. In some embodiments, the discretization of each boundary may be done separately, and may then be concatenated to one “list” of planar triangles to facilitate computation. Each conductivity boundary may comprise the same or different number of triangles. The unknown surface charge density is constant for every triangle and equal to ρᵢ, i=1, . . . , N. Purely parametric time dependence is eliminated by separation of variables. The formulation results in a system of linear equations for unknown induced charges at the boundaries that is solved iteratively. For the n^{th} iteration:

$${ρ}_{i , n} = 2 {ε}_{0} \frac{{σ}_{i n} - {σ}_{o u t}}{{σ}_{i n} + {σ}_{o u t}} {n}_{i} \cdot \{ {E}_{n - 1}^{s} ( {c}_{i} ) + {E}_{i n c} ( {c}_{i} ) \} \qquad ( 1 )$$

$${E}_{n - 1}^{s} ( {c}_{i} ) = - {\sum }_{j = 1}^{N} \frac{{A}_{j} {ρ}_{j , n - 1}}{4 π {ε}_{0}} \frac{{c}_{j} - {c}_{i}}{{\vert {c}_{i} - {c}_{j} \vert}^{3}} \qquad ( 2 )$$

where E_{inc} and E^{s} are the incident field of the coil and secondary field of the charges induced at the interface of any two compartments with conductivities σᵢₙ and σₒᵤₜ, respectively, and ε₀ is the permittivity of vacuum. The conductivities σᵢₙ and σₒᵤₜ are determined based on how the outward normal to the surfaces are defined. In some embodiment, for example, at the GM surface (boundary between GM and CSF) σᵢₙ is the conductivity of GM and σₒᵤₜ is the conductivity of CSF. The iterative solution repeats computation of the E-field from a large ensemble of point charge sources at a large number of target points following Eq. (2). The equations may be iteratively solved using, for example, the Generalized Minimal Residual Method (GMRES). Once the solution converges, the total E-field at any desired location of the space can be determined.

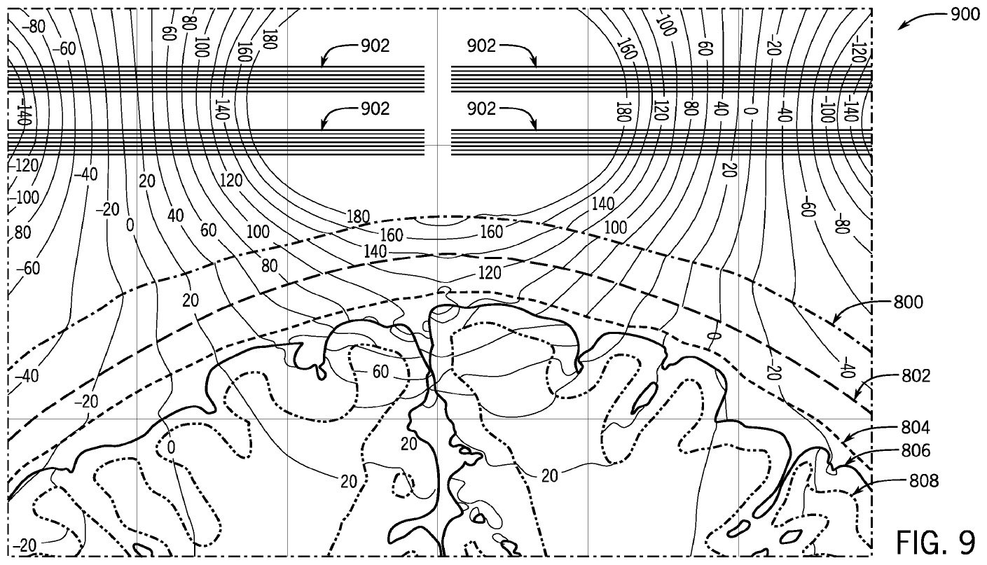

FIG. 9 illustrates an exemplary result of the BEM-FMM calculation of the total E-field (Eₜₒₜ) 900 (in units of V/m), shown at a cross-section in the coronal plane of the head and approximately aligned with the left-right axis of a figure-of-eight coil wings 902, including the total E-field at the skin/scalp 800, skull 802, CSF 804, GM 806, and WM 808. Evaluation of the E-fields caused by TMS coil 902, calculated using BEM-FMM described above, was performed on a current-generation server with 128 GB of memory and required up to 20 seconds to compute. This is insufficient for typical neuronavigation systems which may, for example, have frame rates between 5-15 Hz.

According to some embodiments of the present technology, a stationary basis set may be used to approximate arbitrary surface current distributions. The incident E-field of a moving coil can be matched with a weighted sum of the incident fields of stationary sources (basis set). Matching the incident E-field of the moving coil to the weighted sum of the incident E-fields of the stationary sources will result in the total E-fields (of the coil and of the basis set) being identical, because the solution is determined by the incident E-field and tissue conductivity boundaries (see Eqs. (1) and (2)).

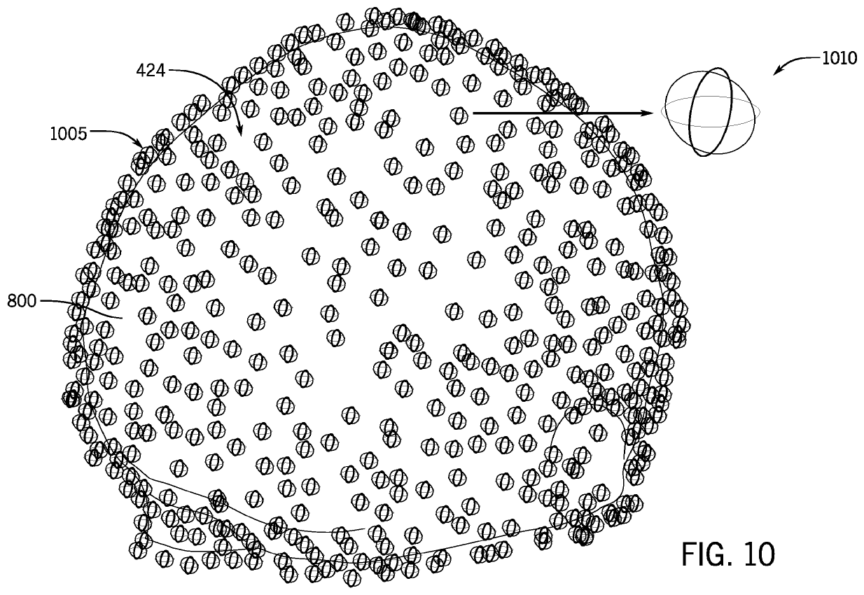

Referring to FIG. 10, in some embodiments, a (stationary) dipole basis set may be created such that it produces a flexible way to approximate the incident E-field of electromagnetic coils. In some embodiments, the dipole basis set may comprise multiple sets of three Cartesian (orthogonal) magnetic dipoles 1010 located at various positions of a discretized surface 1005 (referred to as “dipole surface” herein). The incident E-field patterns of orthogonal dipoles will also be orthogonal. The discretized surface 1005 may comprise a surface of or surrounding the boundary model 424, e.g. surrounding the head. In some embodiments, the dipole surface 1005 may comprise a sphere or other geometric surface. In some embodiments, the dipole surface 1005 may comprise a surface that conforms to the head and is at or some distance (e.g., between about 0.1 mm and 7 mm) away from the boundary model 424. In some embodiments, the dipole surface 1005 may be between about 2 mm and about 5 mm away from the boundary model 424. In an embodiment, referring to FIG. 10, the dipole surface 1005 comprises a surface that conforms to the boundary model 242 at a distance of 2 mm from the skin/scalp 800. In some embodiments, the dipole surface 1005 may be within the head model (e.g., within the scalp surface), but in some cases may lead to increased numerical errors. In some embodiments, the dipole sets may be positioned at each vertex of the discretized dipole surface 1005 (e.g., vertices of the triangles). In some embodiments, the dipole basis set may comprise any 3D arrangement of dipoles not restricted to a surface, which may facilitate an extended range of electromagnetic coil 108 movement with accurate approximations.

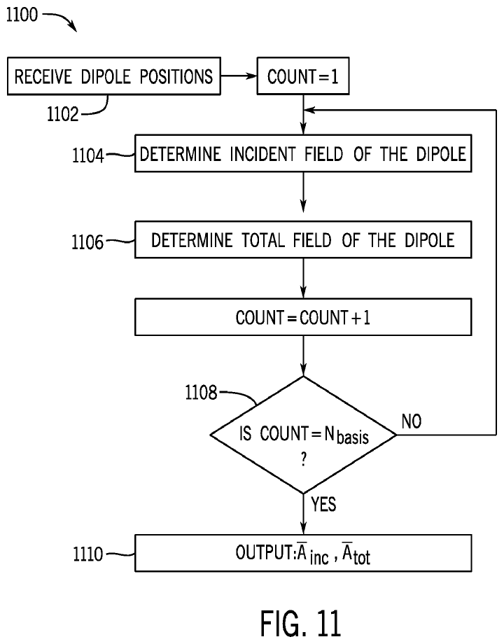

FIG. 11 illustrates an exemplary embodiment of a process for calculating (determining) a Magnetic Stimulation Profile 1100 for a subject based on the dipole basis set. The MSP comprises an indication of the incident E-field of the dipole basis set and an indication of the total E-field of the dipole basis set. The MSP determination process 1100 may be performed by a computer system, for example using E-field determination module 204 and/or the neuronavigation system 202, or other computer system. The MSP determination process 1100 determines the incident E-fields and total E-fields within the boundary model 424 caused by the dipole basis set. The dipole positions may be input, received, or otherwise retrieved 1102 (e.g., from memory such as data storage 212), and for each dipole (N_{basis}=number of dipoles in basis set), the incident E-field of the dipole is determined 1104 for one or more of the conductivity boundaries, and then the total E-field of the dipole is determined 1106 for the one or more conductivity boundaries. Once this has been completed for all dipoles 1108, the process 1100 outputs 1110 the incident E-fields for all dipoles on the one or more conductive boundaries, e.g. as a matrix Ā_{inc}, and the total E-fields for all dipoles on the one or more conductivity boundaries, e.g. as a matrix Āₜₒₜ. Outputting 1110 may comprise storing the results in a memory (e.g., data store 212) or any other suitable method of electronically communicating the results. The output of this process, comprising Ā_{inc} and Āₜₒₜ, may be referred to as the Magnetic Stimulation Profile (MSP) of the subject.

In some embodiments, each column of Ā_{inc} may correspond to the incident field from a dipole computed at the conductivity boundary surfaces. In some embodiments, Ā_{inc} may comprise values for all conductivity boundaries (or a selection of the conductivity boundaries). For example, each column of Ā_{inc} may contain the incident E-fields of one dipole determined at all (or a selection) of the conductivity boundaries. The incident field of a dipole may be a vector having three Cartesian components ((Ex, Ey, Ez)), and all three components may be concatenated. In an embodiment, for a total of N dipole locations and a total of M total surface triangle elements, the size of Ā_{inc} would be M×3N.

In accordance with the above, in some embodiments the incident fields of the dipole basis vector with unit amplitude are computed for the locations of the dipoles and assembled in the matrix Ā_{inc}. The magnetic dipoles 1010 are equivalent to small fictitious current loops. For each dipole, a unit of current Ampere/second can be assigned as it is the current rate-of-change that determines the E-field that is also in the units of V/m. The values of the dipole amplitudes can be normalized in any suitable manner.

The incident E-field of each dipole may be determined 1104 by any suitable method. In some embodiments, the incident E-field of each dipole may be calculated using a Fast Multilevel Multipole method. The total E-field of each dipole comprises the combination of the incident E-field of the dipole and the secondary E-field caused by induced charges, as described above. Calculating the total E-field 1106 may comprise any suitable method for calculating the secondary E-fields, for example the BEM-FMM method as described above with respect to Eqs. (1) and (2). In some embodiments, the MSP may comprise the determined secondary E-fields instead of or in addition to the total E-fields. In embodiments where the MSP comprises secondary E-fields, the total E-fields may be determined by combining the induced and secondary E-fields, which may be pre-calculated or done in real-time. In some embodiments, the MSP may comprise the pseudo-inverse of the dipole basis set (W, described in more detail below) instead of or in addition to the incident E-fields for all dipoles on the one or more conductive boundaries.

Any suitable number of dipoles may be used for the dipole basis set, for example 1500 dipoles (three orthogonal dipoles at 500 locations on the dipole surface), 3000 dipoles (three orthogonal dipoles at 1000 locations on the dipole surface), 4500 dipoles (three orthogonal dipoles at 1500 locations on the dipole surface), and the like. Increasing the number of dipoles can reduce the numerical errors but requires more computation time. A smaller dipole basis set can increase numerical instability on matching E-fields. In some embodiments, the dipole basis set may comprise about 200 to about 2500 dipole locations (about 600 to about 7500 total dipoles). In some embodiments, the dipole basis set may comprise about 500 to about 1000 dipole locations (about 1500 to about 3000 total dipoles), which may achieve both accurate E-field estimation and computational efficiency. One dipole solution may take several seconds to calculate and calculating the entire MSP may take several hours. For example, using a current-generation computer server, it may take approximately 20 seconds to calculate a single dipole solution and approximately 18 hours to calculate the entire MSP for 3000 dipoles in the basis set. Advantageously, the MSP is independent of the electromagnetic coil geometry, need only be calculated once per subject, and can be calculated prior to use by the E-field determination module 204. For example, the MSP may be pre-calculated (predetermined) prior to use in a neuroscience or clinical setting (e.g., prior to application of TMS). By way of non-limiting example, a predetermined MSP for a model having 750,000 elements total (all surfaces) and 1000 dipole locations (3000 dipole basis functions), wherein the MSP comprises the pseudo-inverse of the dipole basis set (W) and Āₜₒₜ, may take approximately 35 GB of memory.

In some embodiments, linear combinations of dipoles can be used as basis functions. In some embodiments, any complete set of surface or volume current distributions may be used as a basis set. For example, this may correspond to changing from one basis set to another by means of a linear (matrix) transformation.

In some embodiments, the incident E-field of the electromagnetic coil is matched to the E-field of the dipole basis set. As noted above, matching the incident E-field of the electromagnetic coil to the incident E-field of the dipole basis set will result in the total E-fields being the same. For arbitrary dipole amplitudes m the incident field at any surface of interest is given by:

$${E}_{i n c}^{d} = {A}_{i n c} m , \qquad ( 3 )$$

where A_{inc} is a sub-matrix of Ā_{inc} corresponding to a surface of interest and E_{inc}^{d} comprises the resulting incident E-fields of the dipoles. The surface of interest can be one of the conductive boundary surfaces (e.g., WM, GM, etc.), a portion of one of the conductivity boundary surfaces, a combination of several surfaces, interpolated in-between surfaces (e.g., surface somewhere between GM and WM), and so on. The selection of the surface of interest may be made by a user or operator, for example an operator of the integrated TMS system 200. The selection may be based on where the fields will be ideally matched and the computational speed requirements—the more points used, the longer the computation may take.

Given an incident field pattern E_{inc} of the electromagnetic coil that is a vector defined at each location of the surface of interest (or “matching surface”), basis coefficients (\hat{m}) can be found to match A_{inc} to E_{inc}. Any suitable method may be used to determine the basis coefficients (\hat{m}). In some embodiments, basis coefficients may be found as the regularized minimum-norm solution:

$$W = {( {A}_{i n c}^{T} {A}_{i n c} + {λ}^{2} I )}^{- 1} {A}_{i n c}^{T} \qquad ( 4 )$$

wherein W is the pseudo-inverse of the dipole basis set, I is the identity matrix, and λ is the regularization parameter described below, and

$$\hat{m} = W {E}_{i n c} \qquad ( 5 )$$

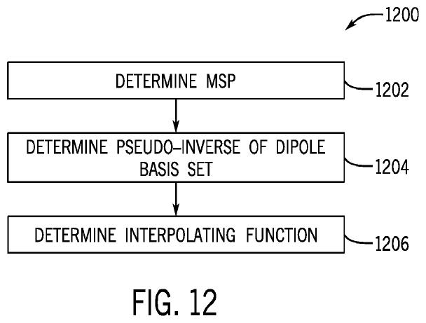

FIG. 12 illustrates an exemplary embodiment of a process for pre-calculating inputs 1200 for the E-field determination module 204, which may be performed by a computer system, for example using E-field determination module 204 and/or the neuronavigation system 202, or other computer system. After the MSP is determined 1100 as discussed above, the pseudo-inverse of the dipole basis may be determined 1202 using matrix A_{inc} and the predetermined EFM 422 as in Eq. (4). In some embodiments, multiple pseudo-inverses of the dipole basis may be determined 1202, one for each surface of interest (e.g., a pseudo-inverse of the dipole basis set for each of the conductivity boundaries of interest). Advantageously, the pseudo-inverse of the dipole basis set need only be calculated once per subject per surface of interest, and can be calculated prior to use by the E-field determination module 204. For example, the pseudo-inverse may be pre-calculated (predetermined) prior to (including at the beginning of) a real-time application of TMS.

The regularization parameter λ may be selected such that it results in a stable estimate: if a small amount of numerical noise is added to the E-field to be matched, the dipole amplitudes should not change significantly. In an example, the regularization parameter may be set to approximately 30% of the total number of dipole basis functions, e.g., the 1000^{th} highest singular value in the matrix Ā_{inc} for a total number of 3000 dipole basis functions. In an exemplary embodiment, λ may be selected in the range of about 5e⁻¹² to about 5e⁻¹⁰. Known methods may be used to set the regularization parameter, for example L-curve method, cross-validation, and the like.

The process for pre-calculating inputs 1200 may further comprise pre-calculating the EFM for the electromagnetic coil, which may comprise calculating the incident E-field on grid points and determining an interpolation function F_{inc}^{g}, as described in more detail above. Pre-calculating the EFM 1204 may occur at any time prior to use by the E-field determination module 204, for example before or after either of the steps of determining MSP 1100 or determining the pseudo-inverse 1202. Each of the steps of the pre-calculation process 1200 may be performed at any time prior to use by the E-field determination module 204, for example hours, days, weeks, months, or years prior to such use.

Referring to FIG. 13, in some embodiments, a method for real-time E-field determination 1300 may comprise receiving location information 1302 of an electromagnetic coil, such as a TMS coil, aligning 1304 the boundary model 424 and EFM 422, determining the incident E-field of the coil 1306 on the surface of interest, determining the matching basis functions coefficients 1308, determining an approximation of the total E-field of the coil 1310 at the surface of interest, and outputting the determined E-field approximation 1312. The E-field determination method 1300 may be performed by a computer system, for example using E-field determination module 204 and/or the neuronavigation system 202, or other computer system.

Receiving location information 1302 may comprise receiving or otherwise retrieving information indicating the position of the electromagnetic coil. The position information may be received from the neuronavigation system 202, retrieved from memory such as data storage 212, or obtained by any other suitable method. The method for real-time E-field determination 1300 may repeat upon a change of position of the electromagnetic coil.

Aligning 1304 the boundary model 424 and EFM 422 may comprise any suitable method of accurately relating the boundary model 424 and EFM 422 positions, for example in three-dimensional space. Aligning 1304 the boundary model 424 and EFM 422 facilitates calculating the incident E-field of the coil (E_{inc}) at any location in the boundary model 424, based on the location of the coil relative to the subject. For example, the incident E-field may be calculated at a surface of interest as described above.

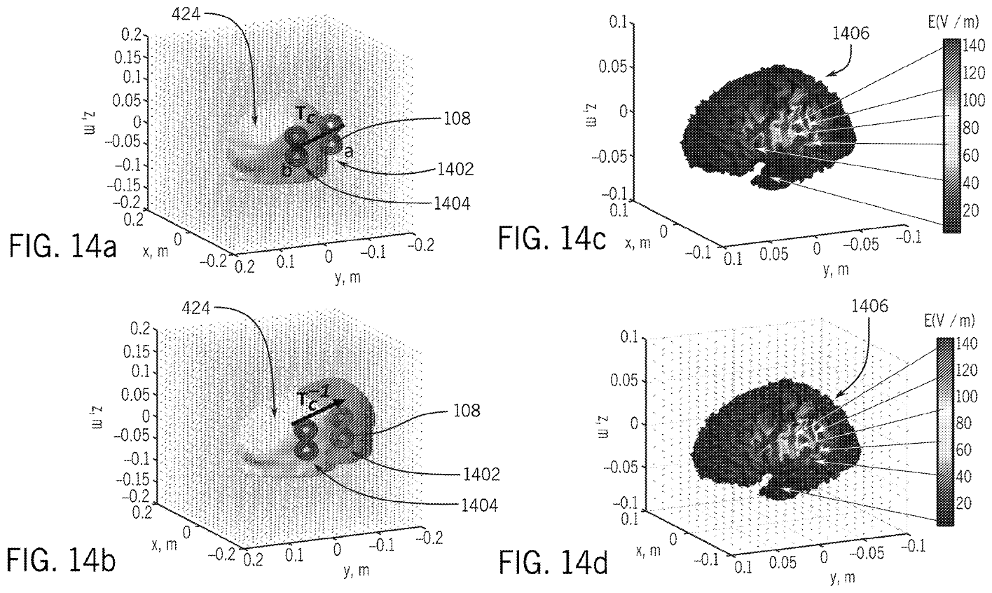

Referring to FIG. 14(a), the physical coil 108 may be moved from a first location 1402 to a second location 1404 about the subject, as described by a transformation matrix T_{c}. T_{c} may comprise a rigid transformation matrix of the form:

$${T}_{c} = [ \begin{matrix}{R}_{c} & {T}_{0} \\ 0 & 1\end{matrix} ] , \qquad ( 6 )$$

wherein R_{c} comprises the 3D rotation matrix and T₀ comprises the translation vector from the first (previous) location 1402 of the coil to a second (current) location of the coil according to the received 1302 location information. In some embodiments, aligning 1304 the boundary model 424 and EFM 422 may comprise transforming the EFM 424 using the matrix T_{c}. The E-field of an electromagnetic coil is translationally (and rotationally) invariant in free space and consequently transforming the coil along a certain direction and rotation is equivalent to moving the boundary model 424 in the opposite direction. Referring to FIG. 14(b), in some embodiments, aligning 1304 the boundary model 424 and EFM 422 may comprise keeping the EFM stationary while transforming the boundary model using T_{c}⁻¹.

Referring again to FIG. 13, determining the incident E-field (E_{inc}) of the coil 1306 on the surface of interest may comprise any suitable method for calculating the E-field of the coil 1306 on a surface of interest of the boundary model 424, using the aligned 1304 EFM 422 and boundary model 424. In some embodiments, E_{inc} may be determined by using the interpolating function F_{inc}^{g} on the surface of interest (the “matching surface”). As noted above, surface of interest can be one of the conductive boundary surfaces (e.g., WM, GM, etc.), a combination of several surfaces, interpolated in-between surfaces (e.g., surface somewhere between GM and WM), and so on. For example, briefly referring again to FIG. 14, FIG. 14(d) illustrates an example of the determined incident E-field (E_{inc}) of the coil on the WM surface 1406 of the boundary model 424 using an interpolating function (F_{inc}^{g}), which compares accurately to FIG. 14(c) which shows the numerically accurate “ground truth” incident E-field of the coil on the WM surface 1406 calculated using a full BEM-FMM method.

Determining the matching basis functions coefficients 1308 may comprise any suitable method for matching the incident E-field of the dipole basis set on the surface of interest to the determined 1306 incident E-field (E_{inc}) of the coil on the surface of interest. In some embodiments, a set of basis function coefficients may be determined that will make the dipole basis set incident E-field identical (or approximately identical) to the incident E-field of the coil, on the surface of interest. In some embodiment, the basis function coefficients \hat{m} may be determined using the determined 1202 pseudo-inverse of the dipole basis set W according to Eq. 5 above. The total E-field of the coil on the surface of interest may then be approximated 1310 as:

$${E}_{t o t}^{d} = {A}_{t o t} \hat{m} , \qquad ( 7 )$$

where Eₜₒₜ comprises the total E-fields of the dipole basis set on the surface of interest, and Aₜₒₜ is a sub-matrix of Āₜₒₜ corresponding to a surface of interest. In some embodiments, determining the matching basis function coefficients 1308 may be done on a first number of points on the surface of interest, and determining the total E-field approximation 1310 may be done on a second number of points on the surface of interest. By way of non-limiting example, 60,000 points on the WM surface may be used to determine matching basis function coefficients, and total E-field may be approximated at 116,000 points on the WM.

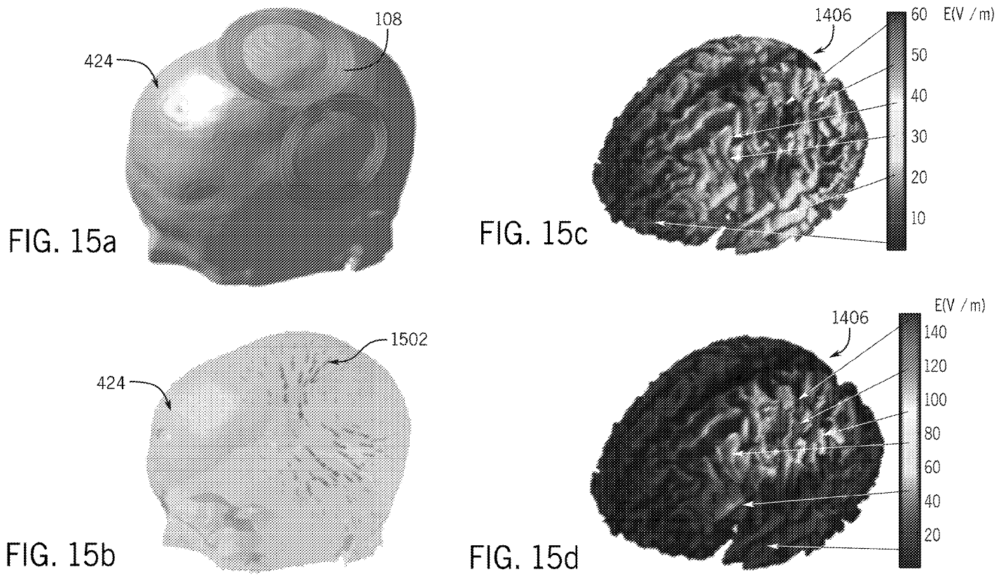

For further example, FIG. 15(a) illustrates a figure-of-eight electromagnetic coil 108 placed about the boundary model 424, FIG. 15(b) illustrates the determined dipole basis set coefficients (\hat{m}) 1308, FIG. 15(c) illustrates the determined incident E-field of the coil 1206 on the WM surface, and FIG. 15(d) illustrates the determined approximation of the total E-field 1310 at the WM surface.

After the total E-field of the coil on the surface of interest is approximated (Eₜₒₜ^{d}) 1310, the approximation may be output 1312. Outputting 1312 the approximated total E-field may comprise storing the approximation in a memory (e.g., data store 212), providing the approximation to the graphical rendering module 206, providing the approximation to the neuronavigation system 202, or any other suitable method of electronically communicating the approximation. In some embodiments, outputting 1312 the approximated total E-field may comprise making the approximated total E-field (Eₜₒₜ^{d}) available at an output of the E-field determination system 204, for example at an output of the processing unit 302, as an electronic data file.

The “linearized” approach described herein eliminates the need for any iterative solver to be applied after the boundary model 424 and MSP 426 of the subject, and EFM of the coil 422, have been determined and allows for real-time visualization of the total E-field of a moving TMS coil on the subject. The approach also decouples the electromagnetic coil model and the MSP, enabling both to be independently pre-calculated, stored, and subsequently linked through incident fields as described above. The systems and methods described herein facilitate real-time approximation of total E-fields on a surface of interest, caused by an electromagnetic coil, with relative maximum error of approximately 5% for E-field amplitude, and spatial distribution patterns having a correlation greater than about 98%. In an exemplary embodiment, on a current-generation server, computation of the total E-field approximation took about 100 ms on a cortical surface mesh with 120,000 triangular elements (also referred to as “facets”).

In some embodiments, increases in computational speed may be obtained by down-sampling the surface meshes. Advantageously, the MSP-basis approach described above allows down-sampling after computing the E-field distributions with high numerical accuracy to avoid accumulation of errors. Further, the E-fields can be re-sampled to be defined on the surface vertices instead of faces (as there are more faces than vertices), resulting in reduced matrix sizes and further reductions in the time needed for the real-time process 1300. The number of dipole basis functions can be also reduced by applying the Singular Value Decomposition (SVD) to A_{inc}. With this approach, the spatially overlapping local dipole basis functions can be compressed to a smaller number of orthonormal global basis functions comprising of the singular vectors. This significantly reduces the computational cost of calculating the MSP of each subject without losing significant information as the SVD basis set can be constructed prior to determining Aₜₒₜ.

It will be appreciated by those skilled in the art that while the disclosed subject matter has been described above in connection with particular embodiments and examples, the present disclosure and the claims of the present disclosure are not necessarily so limited, and that numerous other embodiments, examples, uses, modifications and departures from the embodiments, examples and uses are intended to be encompassed by the claims attached hereto. The entire disclosure of each patent and publication cited herein is hereby incorporated by reference, as if each such patent or publication were individually incorporated by reference herein.

Various features and advantages of the various aspects presented in the present disclosure are set forth in the following claims.

1. An E-field determination system for an electromagnetic coil positioned about a subject having one or more conductivity boundaries, the system comprising:
a memory configured to store therein:
a predetermined electromagnetic coil E-field map;
a predetermined boundary model associated with the subject, wherein the boundary model comprises a model of a surface of a first conductivity boundary of the subject; and
a predetermined Magnetic Stimulation Profile (MSP) associated with the subject, wherein the MSP comprises:
the incident E-field at a first surface of interest (A_{inc}) caused by a basis set of magnetic dipoles; and
the total E-field at the first surface of interest (Aₜₒₜ) caused by the basis set of magnetic dipoles; and
a processor communicatively coupled with the memory and configured to:
(a) receive a location information of the electromagnetic coil;
(b) align, based on the received location information, the predetermined boundary model with the predetermined electromagnetic coil E-field map;
(c) determine the incident E-field (E_{inc}) of the electromagnetic coil at the first surface of interest based on the aligned predetermined electromagnetic coil E-field map and predetermined boundary model;
(d) determine basis function coefficients (\hat{m}) that match the incident E-field (A_{inc}) of the basis set of magnetic dipoles to the determined incident E-field of the electromagnetic coil (E_{inc}) at the first surface of interest;
(e) determine an approximation (Eₜₒₜ^{d}) of the total E-field of the electromagnetic coil at the first surface of interest, wherein:
Eₜₒₜ^{d}=Aₜₒₜ\hat{m}; and
(f) output the approximation (Eₜₒₜ^{d}) of the total E-field of the electromagnetic coil at the first surface of interest.

2. The E-field determination system of claim 1, wherein the system is configured to repeat (b) to (e) for changing location information at least five times in a second.

3. The E-field determination system of claim 1, wherein:
the predetermined electromagnetic coil E-field map comprises an interpolating function (F_{inc}^{g}); and
determining the incident E-field (E_{inc}) of the electromagnetic coil at the first surface of interest comprises using the interpolating function (F_{inc}^{g}) on the first surface of interest.

4. The E-field determination system of claim 1, wherein:
aligning the predetermined boundary model with the predetermined electromagnetic coil E-field map comprises performing transform T_{c}⁻¹ on the boundary model, wherein:
$${T}_{c} = [ \begin{matrix}{R}_{c} & {T}_{0} \\ 0 & 1\end{matrix} ] ,$$
wherein R_{c} comprises the three-dimensional rotation matrix and T₀ comprises the translation vector from a previous location of the electromagnetic coil to a current location of the electromagnetic coil according to the received location information.

5. The E-field determination system of claim 1, wherein the basis function coefficients (\hat{m}) are determined by:
$$\hat{m} = W {E}_{i n c} , wherein W = {( {A}_{i n c}^{T} {A}_{i n c} + {λ}^{2} I )}^{- 1} {A}_{i n c}^{T} ,$$
wherein λ is a regularization parameter.

6. The E-field determination system of claim 1, wherein the surface of interest comprises a conductivity boundary.

7. The E-field determination system of claim 1, wherein the basis set of magnetic dipoles comprises a plurality of sets of three orthogonal magnetic dipoles located on a second surface around the model of the surface of the first conductivity boundary.

8. The E-field determination system of claim 7, wherein the total E-field at the first surface of interest (Aₜₒₜ) caused by the basis set of magnetic dipoles is determined by the summation of the incident E-field at the first surface of interest (A_{inc}) caused by the basis set of magnetic dipoles and a secondary E-field at the first surface of interest (A_{S}) caused by a charge accumulation.

9. The E-field determination system of claim 8, wherein the secondary E-field at the first surface of interest (A_{S}) is determined according to a Boundary Element Method utilizing Fast Multilevel Multipole (BEM-FMM).

10. The E-field determination of claim 1, wherein the MSP comprises:
the incident E-field at a plurality of surfaces of interest (Ā_{inc}) caused by the basis set of magnetic dipoles, wherein A_{inc} is a subset of Ā_{inc}; and
the total E-field at a plurality of surfaces of interest (Āₜₒₜ) caused by the basis set of magnetic dipoles, wherein Aₜₒₜ is a subset of Āₜₒₜ.

11. A method for E-field determination for an electromagnetic coil positioned about a subject having one or more conductivity boundaries, the method comprising:
(a) retrieving, from a memory:
a predetermined electromagnetic coil E-field map;
a predetermined boundary model associated with the subject, wherein the boundary model comprises a model of a surface of a first conductivity boundary of the subject; and
a predetermined Magnetic Stimulation Profile (MSP) associated with the subject, wherein the MSP comprises:
the incident E-field at a first surface of interest (A_{inc}) caused by a basis set of magnetic dipoles; and
the total E-field at the first surface of interest (Aₜₒₜ) caused by the basis set of magnetic dipoles;
(b) receiving, using a processor, a location information of the electromagnetic coil;
(c) aligning, using the processor and based on the received location information, the predetermined boundary model with the predetermined electromagnetic coil E-field map;
(d) determining, using the processor, the incident E-field (E_{inc}) of the electromagnetic coil at the first surface of interest based on the aligned predetermined electromagnetic coil E-field map and predetermined boundary model;
(e) determining, using the processor, basis function coefficients (\hat{m}) that match the predetermined incident E-field at the first surface of interest (A_{inc}) to the determined incident E-field of the electromagnetic coil (E_{inc}) at the first surface of interest;
(f) determining, using the processor, an approximation (Eₜₒₜ^{d}) of the total E-field of the electromagnetic coil at the first surface of interest, wherein:
$${E}_{t o t}^{d} = {A}_{t o t} \hat{m} ;$$
and
(f) outputting the approximation (Eₜₒₜ^{d}) of the total E-field of the electromagnetic coil at the first surface of interest.

12. The method of claim 11, further comprising repeating (c) to (f) for changing location information at least five times in a second.

13. The method of claim 11, wherein:
the predetermined electromagnetic coil E-field map comprises an interpolating function (F_{inc}^{g}); and
determining the incident E-field (E_{inc}) of the electromagnetic coil at the first conductivity surface of interest using the interpolating function (F_{inc}^{g}) on the first surface of interest.

14. The method of claim 11, wherein:
aligning the predetermined boundary model with the predetermined electromagnetic coil E-field map comprises performing transform T_{c}⁻¹ on the boundary model, wherein:
$${T}_{c} = [ \begin{matrix}{R}_{c} & {T}_{0} \\ 0 & 1\end{matrix} ] ,$$
wherein R_{c} comprises the three-dimensional rotation matrix and T₀ comprises the translation vector from a previous location of the electromagnetic coil to a current location of the electromagnetic coil according to the received location information.

15. The method claim 11, wherein determining the basis function coefficients (\hat{m}) comprises:
$$\hat{m} = W {E}_{i n c} , wherein W = {( {A}_{i n c}^{T} {A}_{i n c} + {λ}^{2} I )}^{- 1} {A}_{i n c}^{T} ,$$
wherein λ is a regularization parameter.

16. The method of claim 11, wherein the surface of interest comprises a conductivity boundary.

17. The method of claim 11, wherein the basis set of magnetic dipoles comprises a plurality of sets of three orthogonal magnetic dipoles located on a second surface around the model of the surface of the first conductivity boundary.

18. The method of claim 17, wherein the predetermined total E-field at the first surface of interest (Aₜₒₜ) caused by the basis set of magnetic dipoles comprises the summation of the predetermined incident E-field at the first surface of interest (A_{inc}) caused by the basis set of magnetic dipoles and a predetermined secondary E-field at the first surface of interest (A_{S}) caused by a charge accumulation.

19. The method of claim 18, wherein the predetermined secondary E-field at the first surface of interest (A_{S}) is determined according to a Boundary Element Method utilizing Fast Multilevel Multipole (BEM-FMM).

20. The method of claim 11, wherein the MSP comprises:
the incident E-field at a plurality of surfaces of interest (Ā_{inc}) caused by the basis set of magnetic dipoles, wherein A_{inc} is a subset of Ā_{inc}; and
the total E-field at a plurality of surface of interest (Āₜₒₜ) caused by the basis set of magnetic dipoles, wherein Aₜₒₜ is a subset of Āₜₒₜ.

21. A system for positioning an electromagnetic coil about a subject, the system comprising:
a memory having stored therein:
a predetermined electromagnetic coil E-field map;
a predetermined boundary model associated with the subject, wherein the boundary model comprises a model of a surface of a first conductivity boundary of the subject;
a predetermined Magnetic Stimulation Profile (MSP) associated with the subject;
a processor communicatively coupled with the memory and configured to:
(a) receive a location information of the electromagnetic coil;
(b) align, based on the received location information, the predetermined boundary model with the predetermined electromagnetic coil E-field map;
(c) determine an incident E-field (E_{inc}) of the electromagnetic coil at the first surface of interest based on the aligned predetermined electromagnetic coil E-field map and predetermined boundary model;
(d) determine an approximation (Eₜₒₜ^{d}) of the total E-field of the electromagnetic coil at the first surface using the MSP and the incident E-field; and
(e) generate a report indicating (Eₜₒₜ^{d}) of the total E-field of the electromagnetic coil at the first surface of interest.
